(19) United States
(12) Patent Application Publication
(10) Pub. No.: US 2022/0288456 A1
(43) Pub. Date: Sep. 15, 2022

(54) SYSTEMS AND METHODS FOR DETERMINING A QUALITY OR TYPE OF A PHYSICAL ACTIVITY IN A SPATIAL AREA

(71) Applicant: SIGNIFY HOLDING B.V., EINDHOVEN (NL)

(72) Inventor: ABHISHEK MURTHY, ARLINGTON, MA (US)

(21) Appl. No.: 17/636,114

(22) PCT Filed: Aug. 18, 2020
(86) PCT No.: PCT/EP2020/073102

§ 371 Date: Feb. 17, 2022

Related U.S. Application Data
(60) Provisional application No. 62/889,277, filed on Aug. 20, 2019.

(30) Foreign Application Priority Data
Aug. 29, 2019 (EP) 19194252.3

(51) Int. Cl. A63B 24/00; A63B 71/06; G16H 40/67

(52) U.S. Cl. CPC A63B 24/0062; A63B 71/0622; G16H 40/67; A63B 2225/74; A63B 2230/50; A63B 2024/0068

(57) ABSTRACT

The present disclosure is directed to systems and methods of determining a quality or type of a physical activity such as a fitness center. Generally, in one aspect, a controller for determining a quality or type of a physical activity performed by one or more persons in a spatial area is provided. The controller is configured to: (i) determine an activity pattern based on infrared energy detected in a spatial area by one or more thermopile sensors; (ii) determine a difference between said activity pattern 110 and a nominal pattern; (iii) determine a quality or type of a physical activity performed by said one or more persons based on said difference; and (iv) provide an output signal based on said quality or type of said physical activity in said spatial area. The quality of said physical activity may be a safety determination. The type of said physical activity may be a physical activity determination.

### FIELD OF THE INVENTION

The present disclosure is directed to systems and methods of determining a quality or type of a physical activity of an individual in a spatial area, for example, a fitness center.

### BACKGROUND

Fitness centers often include large indoor areas with a variety of exercise equipment. Much of this exercise equipment, such as weight machines, lacks any electrical components or connectivity features for monitoring usage by an individual. Consequently, the individual may be unknowingly using the equipment in a hazardous manner, threatening the individual's safety.

Further, fitness centers may also have enclosed rooms in which different group exercise or physical activities are performed. These activities may range from high intensity exercises like spinning and Zumba®, to low intensity exercises like yoga and Tai chi. The ambient conditions required for each activity may be quite different. Specifically, the humidity and the lighting in the room may desired to be maintained according to the physical activity performed in the room. For instance, Zumba® entails strenuous physical activity requiring aggressive humidity control. Further, Zumba® and yoga may utilize significantly different lighting schemes. As fitness center rooms typically lack the equipment to monitor the activity performed within, the ambient conditions within such spatial areas must be controlled manually.

Accordingly, there is a continued need for systems and methods for monitoring physical activity of individuals in spatial areas such as fitness centers and other spaces.

### SUMMARY OF THE INVENTION

The present disclosure is directed to systems and methods of determining a quality or type of a physical activity in a spatial area. In particular, an aspect of the present disclosure is directed to generating a safety determination of physical activities performed by individuals in a spatial area. A further aspect of the present disclosure is directed to determining a type of activity performed by two or more persons in a spatial area.

Generally, in one aspect, a controller for determining a quality or type of a physical activity performed by one or more persons in a spatial area is provided. The controller is configured to: (i) determine an activity pattern based on infrared energy detected in a spatial area by one or more thermopile sensors; (ii) determine a difference between said activity pattern and a nominal pattern; (iii) determine a quality or type of a physical activity performed by said one or more persons based on said difference; and (iv) provide an output signal based on said quality or type of said physical activity in said spatial area. The one or more thermopile sensors may include one or more single-pixel thermopile sensors. The one or more thermopile sensors may include one or more multi-pixel thermopile sensors. The nominal pattern may be stored in a memory of the controller.

According to an example, the quality of the physical activity may be a safety determination.

According to an example, the nominal pattern may represent a person safely performing said physical activity in said spatial area.

According to an example, exceeding a deviation limit by said difference may result in a safety determination of unsafe.

According to an example, the output signal may be configured to warn said person of unsafe physical activity.

According to an example, the type of the physical activity may be a physical activity determination.

According to an example, the nominal pattern represents a type of physical activity performed by two or more persons in the spatial area.

According to an example, conforming to a deviation limit by the difference results in a type of physical activity determination corresponding to the type of physical activity represented by the nominal pattern.

According to an example, the output signal is configured to program one or more spatial area controls according to said type of physical activity.

Generally, in another aspect, a system for determining a quality or type of a physical activity performed by one or more persons in a spatial area. The system includes one or more luminaires arranged to illuminate said spatial area. The system further includes a controller configured to: (i) determine an activity pattern based on infrared energy detected in a spatial area by one or more thermopile sensors; (ii) determine a difference between said activity pattern and a nominal pattern; (iii) determine a quality or type of a physical activity performed by said one or more persons based on said difference; and (iv) provide an output signal based on said quality or type of said physical activity in said spatial area.

Generally, in another aspect, a method for determining a quality or type of a physical activity performed by one or more persons in a spatial area is provided. The method includes: (i) determining an activity pattern based on infrared energy detected in a spatial area by one or more thermopile sensors; (ii) determining a difference between said activity pattern and a nominal pattern; (iii) determining a quality or type of a physical activity performed by said one or more persons based on said difference; and (iv) providing an output signal based on said quality or type of said physical activity in said spatial area. The method may further include storing said nominal pattern in a memory of said controller.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a schematic of a controller for determining a quality or type of a physical activity performed by one or more persons in a spatial area according to an embodiment of the present disclosure.

FIG. 2 is an example activity pattern illustrating the pattern characteristics of step height, step duration, and inter-arrival time between steps.

FIG. 3 is a schematic of a system for determining a quality or type of a physical activity performed by one or more persons in a spatial area according to an embodiment of the present disclosure.

FIG. 4 is another example activity pattern measured by a single-pixel thermopile.

FIG. 5 is an example activity pattern measured by a multi-pixel thermopile.

FIG. 6 is a block diagram of a method for determining a quality or type of a physical activity performed by one or more persons in a spatial area

### DETAILED DESCRIPTION OF EMBODIMENTS

The present disclosure describes various embodiments of systems and methods of determining a quality or type of a physical activity in a spatial area such as fitness center. More generally, Applicant has recognized and appreciated that it would be beneficial to monitor physical activity performed by one or more persons in a spatial area where physical activity occurs. Exemplary goals of utilization of the present disclosure include improved patron safety, reduction in fitness center heating, ventilation, and air conditioning (HVAC) energy consumption, and greater ease of use of fitness center HVAC systems.

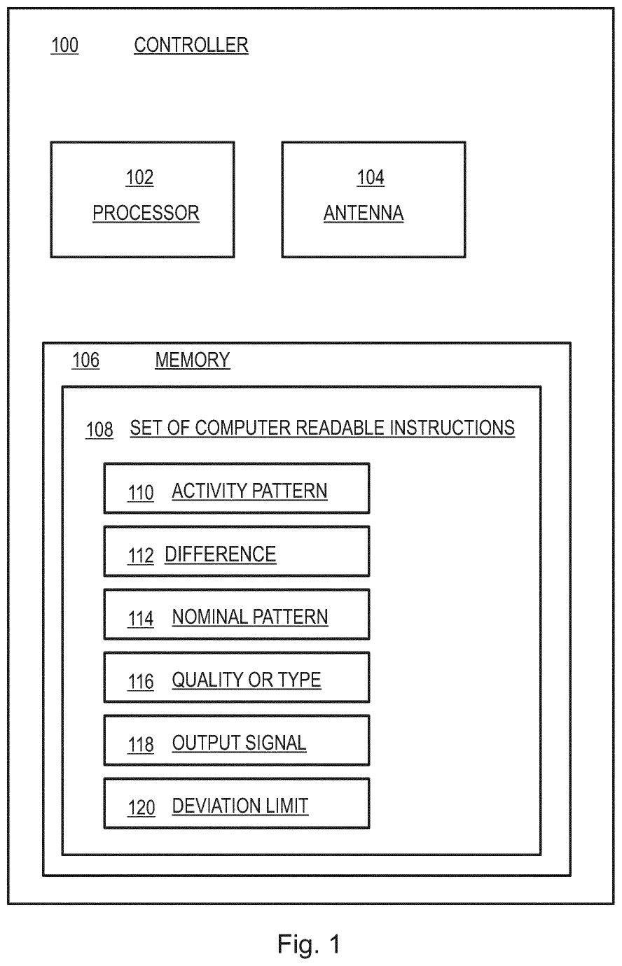

Referring to FIG. 1, a controller 100 for determining a quality or type 116 of a physical activity performed by one or more persons in a spatial area is provided. The controller 100 may include a processor 102, an antenna 104, and a memory 106. The memory 106 may be a non-transitory computer-readable media. The term “controller” is used herein generally to describe various apparatus relating to the operation of one or more luminaires 210. A controller 100 can be implemented in numerous ways (e.g., such as with dedicated hardware) to perform various functions discussed herein. A controller 100 that employs one or more processors 102 may be programmed using software to perform various functions discussed herein. A controller 100 may be implemented as a combination of dedicated hardware to perform some functions and a processor 102 (e.g., one or more programmed microprocessors and associated circuitry) to perform other functions.

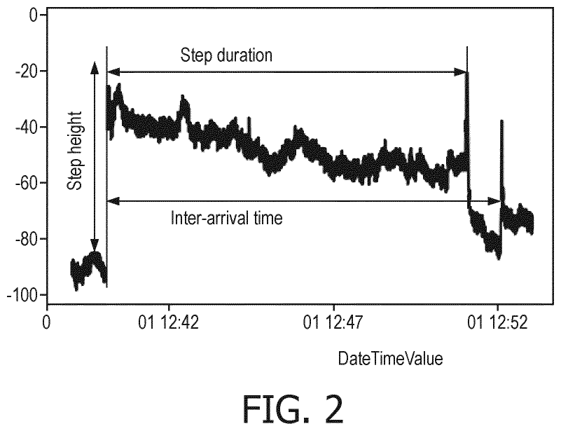

The controller 100 may be configured to determine an activity pattern 110 based on infrared energy detected in a spatial area by one or more thermopile sensors 220. The thermopile sensor 220 produces a signal, or activity pattern 110, corresponding to the temperature of a detected object. This activity pattern 110 may be used to represent a person's motion in a monitored spatial area over a period of time. For example, if a person is standing relatively still for a period of time, the infrared energy detected by the thermopile sensor 220 may be relatively constant and the activity pattern 110 produced by the thermopile sensor 220 may be relatively flat. However, if this person begins to perform a repetitive exercise (such as pull-ups, for example), the thermopile sensor 220 will detect a change in infrared energy as the person moves relative to the thermopile sensor 220. Accordingly, the thermopile sensor 220 will produce an activity pattern 110 showing regular increases and decreases in temperature, corresponding to the person's distance from the sensor. The activity pattern 110 may be characterized according to a variety of pattern features. These features may include, but are not limited to, step height (the change in temperature from peak to trough), step duration (the change in time from peak to trough), inter-arrival time between steps (the change in time between the beginning of a first step and the beginning of a second step), and step slope (the change in temperature during a step). An example activity pattern 110 is shown in FIG. 2, and described in further detail below.

The thermopile sensor 220 may be a single-pixel thermopile sensor to generate an activity pattern 110 based on temperatures measured at a single point in space. The thermopile sensor 220 may be a multi-pixel thermopile sensor to generate an activity pattern 110 based on temperatures measured over an array of points in space. Examples of embodiments utilizing different varieties of thermopile sensors 220 may be found below.

The controller 100 may be further configured to determine a difference 112 between the activity pattern 110 and a nominal pattern 114. The nominal pattern 114 is a stored data set used as a baseline to evaluate one or more qualities of the measured activity pattern 110 through comparison. In one example, the nominal pattern 114 represents a person safely performing the physical activity in the spatial area. In another example, the nominal pattern 114 represents a type of physical activity performed by two or more persons in the spatial area. The nominal pattern 114 may be stored in a memory 106 of the controller 100. The controller 100 may also receive the nominal pattern 114 via any appropriate means, including via external memory 106 or wired or wireless network.

The controller 100 may be further configured to determine a quality or type 116 of a physical activity performed by the one or more persons based on the difference 112. The controller 100 may set a deviation limit 120 for determining the quality or type 116 of the physical activity. In one example, exceeding a deviation limit 120 by the difference 112 results in a safety determination of unsafe. In another example, conforming to a deviation limit 120 by the difference 112 results in a physical activity determination corresponding to the type of physical activity represented by the nominal pattern 114. The deviation limit 120 may be designed to focus the determination of quality or type on one or more pattern features, such as step height, step duration, inter-arrival time between steps, and step slope. Focusing on one or more pattern features may improve the accuracy and/or efficiency of the determination.

The controller 100 may be further configured provide an output signal 118 based on the quality or type 116 of the physical activity in the spatial area. In one example, the output signal 118 is configured to warn said person of unsafe physical activity. In another example, the output signal 118 is configured to program one or more spatial area controls according to said type of physical activity.

The memory 106 and the processor 102 may take any suitable form known in their respective arts that is useful for controlling, monitoring, and/or otherwise assisting in the operation of the thermopile sensors 220. Embodiments of processor 102 include, but are not limited to, conventional microprocessors, application specific integrated circuits (ASICs), and field programmable gate arrays (FPGAs). It is to be understood that the controller 100 is shown schematically in FIG. 1 and may include any other components useful for controlling, monitoring, and/or otherwise assisting in the operation of the thermopile sensors 220.

The controller 100 may be associated with one or more storage media or memory, e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors 102 and/or controllers 100, perform at least some of the functions discussed herein. Various storage media may be fixed within a processor 102 or controller 100 or may be transportable, such that the one or more programs stored thereon can be loaded into a processor 102 or controller 100 so as to implement various aspects of the present invention discussed herein. The term “program” as used herein refers in a generic sense to any type of computer code (e.g., software or microcode) that can be employed to program one or more processors 102 or controllers.

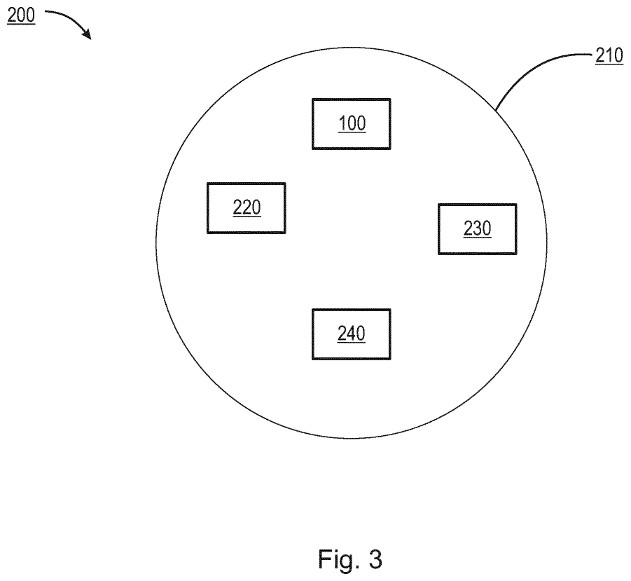

Referring to FIG. 3, a system 200 for determining a quality or type 116 of a physical activity performed by one or more persons in a spatial area is provided. The system 200 includes a luminaire 210 arranged to illuminate the spatial area. The term “luminaire” as used herein refers to an apparatus including one or more light sources of same or different types. A given luminaire may have any one of a variety of mounting arrangements for the light source(s), enclosure/housing arrangements and shapes, and/or electrical and mechanical connection configurations. Additionally, a given lighting unit optionally may be associated with (e.g., include, be coupled to and/or packaged together with) various other components (e.g., control circuitry) relating to the operation of the light source(s). An “LED-based lighting unit” as used herein refers to a lighting unit that includes one or more LED-based light sources as discussed above, alone or in combination with other non LED-based light sources.

The system 200 further may include a controller 100 configured to determine an activity pattern 110 based on infrared energy detected in a spatial area by one or more thermopile sensors 220 arranged in or on the one or more luminaires 210. The controller 100 may be further configured to determine a difference 112 between said activity pattern 110 and a nominal pattern 114. The controller 100 may be further configured to determine a quality or type 116 of a physical activity performed by said one or more persons based on said difference 112. The controller 100 may be further configured to provide an output signal 118 based on said quality or type 116 of said physical activity in said spatial area. The system 200 may further include a transmitter 230 for transmitting information from the system 200 to one or more external wired or wireless devices. The system 200 may further include a light source 240 for illuminating a portion of the spatial area.

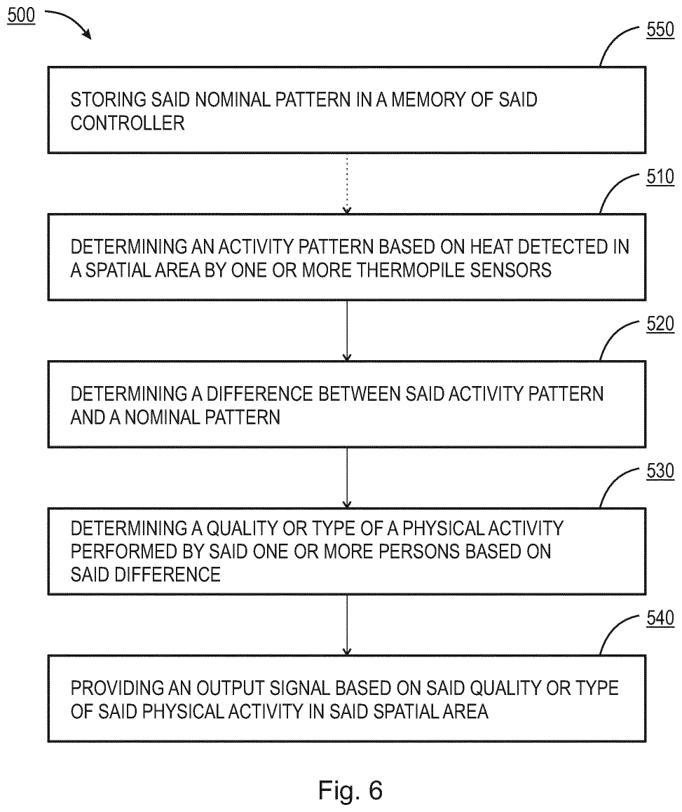

Referring to FIG. 6, a method 500 for determining a quality or type of a physical activity performed by one or more persons in a spatial area is provided. The method 500 may include determining 510 an activity pattern based on infrared energy detected in a spatial area by one or more thermopile sensors. The method 500 may further include determining 520 a difference between said activity pattern and a nominal pattern. The method 500 may further include determining 530 a quality or type of a physical activity performed by said one or more persons based on said difference. The method may further include providing 540 an output signal based on said quality or type of said physical activity in said spatial area. The method may further include storing 550 said nominal pattern in a memory of said controller.

In order to more fully appreciate the features of the disclosed invention, three non-limiting examples are provided below.

### Example 1

In one example of the disclosed invention, a system 200 for detecting unsafe usage of a pull-up machine is provided. As with many types of fitness center fitness equipment, this pull-up machine is a mechanical device with no electrical components or connectivity features. Accordingly, unsafe usage may only be detected through an external monitoring system. In this example, the disclosed invention utilizes a controller 100 with a single-pixel thermopile sensor 220 installed in a luminaire 210 directly above the machine to make a safety determination regarding use of the pull-up machine.

As the user begins to exercise on the pull-up machine, the single-pixel thermopile sensor 220 generates an activity pattern 110 corresponding to the temperatures measured over a period of time. As the user pulls up on the machine and move closer to the sensor, the temperature read by the sensor increases. Inversely, the temperature read by the sensor deceases as the user drops down. Mapping the measured temperatures over time forms an activity pattern 110 which represents the user's movement relative to the thermopile sensor 220. One may appreciate that a repetitive fitness activity, such as a series of pull-ups, would result in a periodic activity pattern 110, with peaks when the user pulls up, and troughs when the user drops down. As can be seen in FIG. 2, the activity pattern 110 may be further characterized using a variety of pattern properties, such as step height (the change in temperature from peak to trough), step duration (the change in time from peak to trough), inter-arrival time between steps (the change in time between the beginning of a first step and the beginning of a second step), and step slope (the change in temperature during a step).

Once the controller 100 captures an activity pattern 110 representing the user's motion on the pull-up machine, the controller 100 will compare the activity pattern 110 to a nominal pattern 114. The nominal pattern 114 represents the expected activity pattern 110 for safe usage of the pull-up machine. One will appreciate that different fitness machines will have different nominal patterns 114 representing different patterns of safe usage. One will also appreciate that one fitness machine may have a wide array of nominal patterns 114 corresponding to different exercises or user body shapes. Further expected variations in safe equipment usage may be accounted for in the nominal pattern 114 and/or in the deviation limit 120 described below.

The controller 100 compares the activity pattern 110 to the nominal pattern 114 and determines a difference 112 between the two patterns. This difference 112 is then compared to a deviation limit 120. The deviation limit may be a preset value representing the degree to which the activity pattern 110 may deviate from the nominal pattern 114 while still representing safe usage. If the difference 112 exceeds the deviation limit 120 at any point in time, the controller 100 makes a safety determination of “unsafe”. Otherwise, the usage of the pull-up machine is determined to be “safe”.

If the controller 100 makes a safety determination of “unsafe”, it may also provide an output signal 118 to one or more devices in order to warn the user of their unsafe physical activity. For example, the output signal 118 may program the light source 240 of the luminaire 210 above the machine to change colors, blink, or any other appropriate configuration. Similarly, the output signal 118 may be transmitted to an audio speaker which produces a sound associated with unsafe activity, such as a loud tone or a verbal “WARNING” message. Further, the output signal 118 may be transmitted to external devices connected via a wired or wireless network, such as a remote monitoring interface. This embodiment may be most useful for large fitness center facilities containing multiple rooms.

### Example 2

In another example of the disclosed invention, a system 200 for detecting a type of physical activity performed by two or more persons is provided. Fitness centers often include enclosed rooms for group fitness activities. These activities may range from high intensity exercises like spinning and Zumba®, to low intensity exercises like yoga and Tai chi. In this example, the disclosed invention utilizes one or more controllers 100 with one or more multi-pixel thermopile sensors 220 installed in one or more luminaires 210 in an enclosed room. This non-limiting example utilizes one controller 100 with one multi-pixel thermopile sensor 220 installed in one luminaire 210.

Similar to Example 1, the multi-pixel thermopile sensor 220 generates an activity pattern 110 corresponding to in-room temperatures of the two or more persons measured over a period of time. Activity patterns 110 with a high degree of variation may correspond to high intensity activities such as spinning and Zumba®. Activity patterns 110 with a low degree of variation may correspond to low intensity activities such as yoga or Tai chi. The activity patterns 110 of this example may be characterized using the same properties as disclosed in Example 1, such as step height, step duration, inter-arrival between steps, and step slope, as shown in FIG. 2.

Once the controller 100 captures the activity pattern 110, the controller 100 will compare the activity pattern 110 to a nominal pattern 114. The nominal pattern 114 represents the expected activity pattern 110 for type of physical activity, such as spinning or yoga. One will appreciate that activity groups of different sizes with have different nominal patterns 114. Further expected variations in a type of physical activity may be accounted for in the nominal pattern 114 and/or in the deviation limit 120.

The controller 100 then determines a difference 112 between activity pattern 110 and nominal pattern 114. This difference 112 is then compared to a deviation limit 120. The deviation limit 120 may be a preset value representing the degree to which the activity pattern 110 may deviate from the nominal pattern 114 while still representing the type of physical activity associated with the nominal pattern. If the difference 112 conforms to the deviation over the time period of the activity pattern 110, the controller 100 determines that the type of physical activity performed by the two or more persons is the type of physical activity represented by the nominal pattern 114.

If the controller 100 determines the type of activity performed by the two or more persons, it may also provide a corresponding output signal 118 to one or more devices. For example, if the controller 100 determines that the two or more persons are engaged in spinning, the controller 100 may program the light source of the luminaire 210 to change colors or strobe 240. Similarly, the output signal 118 may be transmitted to an HVAC controller to adjust the room climate to account for the increase in exertion by the two or more persons. Further, the output signal 118 may be transmitted to external devices connected via a wired or wireless network, such as a remote monitoring interface. The output signal 118 may be adjusted based on additional information received from other sensors, such as humidity and/or audio sensors.

In a related example, the controller 100 could also determine the number of people in the monitored enclosed room by analyzing the difference 112 between activity pattern 110 and nominal pattern 114. In a further related example, each detected person in the monitored closed room could be tracked based on the change in the activity pattern 110 over time.

### Example 3

In another example of the disclosed invention, a system for measuring an amount of usage of an exercise machine is provided. Exercise equipment requires upkeep and maintenance. This upkeep and maintenance may be required at regular time intervals, and also may be required based on the amount of usage the equipment receives. Tracking the usage of the equipment and identifying patterns of use may aid in optimizing equipment maintenance, as well as identifying equipment which may be broken or simply unpopular. In this example, the disclosed invention utilizes a controller 100 with a single-pixel thermopile sensor 220 installed in a luminaire 210 mounted near an exercise machine.

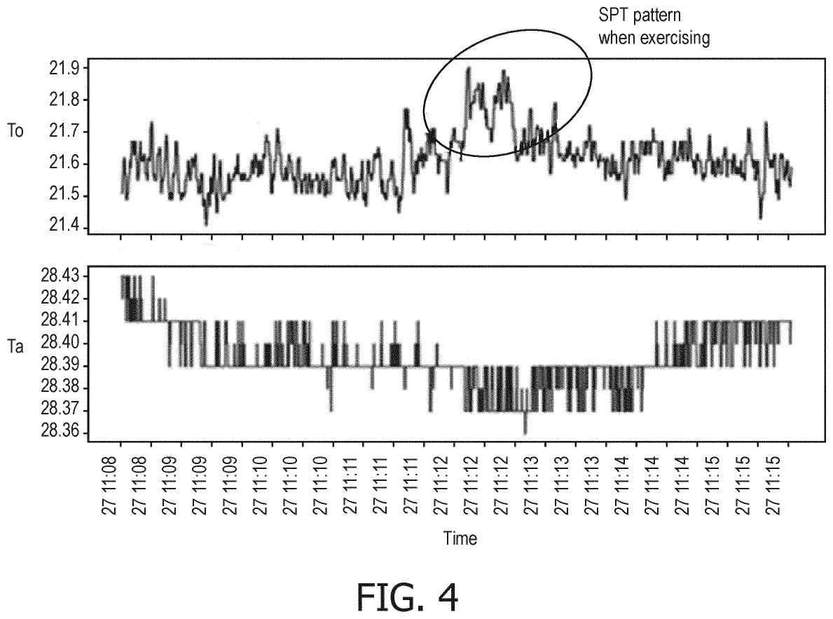

Similar to Examples 1 and 2, the single-pixel thermopile sensor 220 generates an activity pattern 110 corresponding to temperatures measured over an extended period of time, such as weeks or months. Alternatively, a number of discrete activity patterns 110 of shorter periods of time may be generated. Controller 100 analyzes the activity pattern 110 to determine one or more characteristics of the usage of the exercise machine by comparing the activity pattern 110 against one or more nominal patterns 114 of expected usage. For example, FIG. 4 shows an activity pattern 110 of use of an example exercise machine. The nominal patterns 114 may be chosen based upon commissioning data programmed into each individual luminaire 210.

The characteristics of usage may include a total amount of usage over the measurement period, and/or an average amount of time used per user. A high amount of use over the measurement period may be indicative of the machine requiring routine maintenance and upkeep in the near future. Conversely, a low average amount of time used per user may be indicative of faulty equipment. The characteristics of use may then be used to prioritize maintenance activities of the fitness center, such as prioritizing repair of faulty equipment over regular maintenance of functioning equipment.

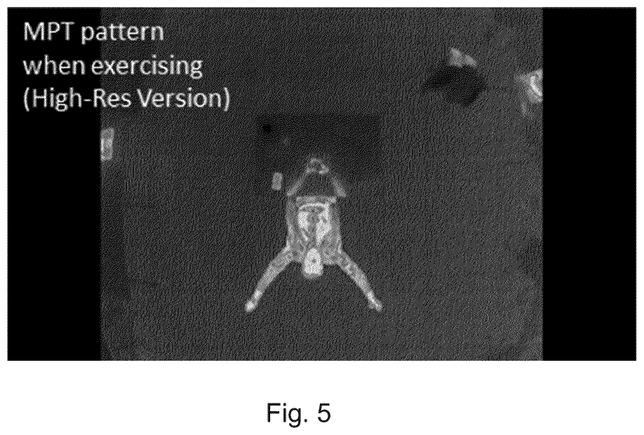

Alternatively, one or more multi-pixel thermopile sensors 220 may be used to generate the activity pattern 110. An example of an activity pattern 100 generated by multi-pixel thermopile sensors is shown in FIG. 5.

In a related example, the activity pattern 110 may simultaneously capture usage of two or more exercise machines (for example, an elliptical and a rowing machine). While each machine has its own set of nominal patterns 114 indicative of use, they may also have a set of nominal patterns 114 indicative of simultaneous use if captured on the same activity pattern 110. Further, the nominal patterns 114 may be used to disambiguate the devices captured by the same activity pattern 110 using a comparative analysis similar to the steps described above.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

It should also be understood that, unless clearly indicated to the contrary, in any methods claimed herein that include more than one step or act, the order of the steps or acts of the method is not necessarily limited to the order in which the steps or acts of the method are recited.

1. A controller for determining a quality of a physical activity performed by one or more persons in a spatial area, said controller configured to:
determine an activity pattern based on infrared energy detected in a spatial area by one or more thermopile sensors;
determine a difference between said activity pattern and a nominal pattern;
determine a quality of a physical activity performed by said one or more persons based on said difference; and
provide an output signal based on said quality of said physical activity in said spatial area,
wherein said quality of said physical activity is a safety determination, and said nominal pattern represents a person safely performing said physical activity in said spatial area, and exceeding a deviation limit by said difference results in a safety determination of unsafe.

2. (canceled)

3. A controller as claimed in claim 1, wherein said one or more thermopile sensors comprise one or more single-pixel thermopile sensors.

4. A controller as claimed in claim 1, wherein said output signal is configured to warn said person of unsafe physical activity.

5. A controller as claimed in claim 1, wherein said quality of said physical activity is a physical activity determination.

6. A controller as claimed in claim 1, wherein said nominal pattern represents a physical activity performed by two or more persons in said spatial area.

7. A controller as claimed in claim 1, wherein conforming to a deviation limit by said difference results in a quality of physical activity determination corresponding to said quality of physical activity represented by said nominal pattern.

8. A controller as claimed in claim 1, wherein said one or more thermopile sensors comprise one or more multi-pixel thermopile sensors.

9. A controller as claimed in claim 1, wherein said output signal is configured to program one or more spatial area controls according to said quality of physical activity.

10. A controller as claimed in claim 1, wherein said nominal pattern is stored in a memory of said controller.

11. A system for determining a quality of a physical activity performed by one or more persons in a spatial area, said system comprising:
one or more luminaires arranged to illuminate said spatial area; and
a controller configured to:
determine an activity pattern based on infrared energy detected in a spatial area by one or more thermopile sensors arranged in or on the one or more luminaires;
determine a difference between said activity pattern and a nominal pattern;
determine a quality of a physical activity performed by said one or more persons based on said difference; and
provide an output signal based on said quality of said physical activity in said spatial area,
wherein said quality of said physical activity is a safety determination, and said nominal pattern represents a person safely performing said physical activity in said spatial area, and exceeding a deviation limit by said difference results in a safety determination of unsafe.

12. A method for determining a quality of a physical activity performed by one or more persons in a spatial area, said method comprising:
determining an activity pattern based on infrared energy detected in a spatial area by one or more thermopile sensors;
determining a difference between said activity pattern and a nominal pattern;
determining a quality of a physical activity performed by said one or more persons based on said difference; and
providing an output signal based on said quality of said physical activity in said spatial area,
wherein said quality of said physical activity is a safety determination, and said nominal pattern represents a person safely performing said physical activity in said spatial area, and exceeding a deviation limit by said difference results in a safety determination of unsafe.

13. A method as claimed in claim 12, wherein the method further comprising storing said nominal pattern in a memory of said controller.
